(19) United States
(12) Patent Application Publication
(10) Pub. No.: US 2006/0165769 A1
(43) Pub. Date: Jul. 27, 2006

(54) Pharmaceutical formulations containing vitamin E TPGS molecules that solubilize lipophilic drugs without significant efflux inhibition, and use of such formulations

(76) Inventors: John Anthony Hyatt, Kingsport, TN (US); George Chester Zima, Kingsport, TN (US); Kevin Joseph Edgar, Kingsport, TN (US); Lisa Navarro, Oak Ridge, NC (US); Claus-Michael Lehr, Saarbrucken-Dudweiler (DE); Eva-Maria Collnot, Ottweiler (DE); Andy Hugh Singleton, Kingsport, TN (US); Christiane Baldes, Saarbrucken (DE); Ulrich Friedrich Schafer, Sulzbach (DE); Michael Wempe, Kingsport, TN (US)

Correspondence Address: William K. McGreevey;Eastman Chemical Company, P.O. Box 511, Kingsport, TN, 37662-5075, US

(21) Appl. No.: 11/238,439

(22) Filed: Sep. 29, 2005

Related U.S. Application Data
(60) Provisional application No. 60/614,891, filed on Sep. 30, 2004.
(60) Provisional application No. 60/691,102, filed on Jun. 16, 2005.

(51) Int. Cl. A61K 9/127; A61K 31/355

(52) U.S. Cl. 424/450; 514/458

(57) ABSTRACT

Compounds and compositions are disclosed for increasing the bioavailability of lipophilic drugs, and more specifically, to solubilizing lipophilic drugs using bioenhancers that cause no efflux inhibition or a desired degree of efflux inhibition. Methods of making and using such compositions are also disclosed.

### CROSS-REFERENCE TO RELATED APPLICATION

This application claims priority to U.S. Provisional Patent Application No. 60/614,891, filed Sep. 30, 2004.

### FIELD OF THE INVENTION

The invention relates to increasing the bioavailability of lipophilic drugs, and more specifically, to solubilizing lipophilic drugs using bioenhancers that achieve a desired degree of efflux inhibition.

### BACKGROUND OF THE INVENTION

Water-soluble vitamin E-active polyethylene glycol esters of tocopheryl acid such as succinates were developed to provide water-soluble molecules having high vitamin E activity via either oral or parenteral administration. Examples include the polyethylene glycol acid succinate of α-tocopherol, known as d-α-tocopheryl polyethylene glycol succinate (TPGS). U.S. Pat. No. 2,680,749 discloses TPGS molecules in which the polyethylene glycols have average molecular weights of 400, 1000, and those varying between 600 and 6000.

TPGS molecules in which the polyethylene glycol chains have an average molecular weight (MW) of about 1000 (TPGS 1000; available from Eastman Chemical Company, Kingsport, Tenn.) are currently used in oral pharmaceutical applications to enhance the bioavailability of various drugs.

Due to the amphiphilic nature of TPGS 1000, incorporating TPGS 1000 into pharmaceutical formulations enhances oral bioavailability by solubilizing some hydrophobic drugs. TPGS 1000 is also believed to influence one or more transporter proteins, one example of which is P-glycoprotein (P-gp), an enzyme that acts as a cellular efflux pump. Therefore, TPGS 1000 may contribute to oral bioavailability enhancement by influencing efflux of some drugs.

Although efflux inhibition results in increased oral bioavailability of certain drugs, it is also desirable in some circumstances to avoid efflux inhibition or to control the degree to which efflux inhibition occurs. For example, administration of an efflux inhibitor in a pharmaceutical formulation may result in the need for additional testing to determine whether the efflux inhibitor has an impact on the oral bioavailability (absorption, metabolism, distribution, or clearance) of other coadministered drugs or dietary substances. Controlling the degree of efflux inhibition can also be desirable where a number of substances subject to efflux need to be considered. It would be an advance in the art to provide pharmaceutical formulations that contain a solubility-enhancing TPGS molecule to enhance the bioavailability of lipophilic drugs, while avoiding efflux inhibition altogether or achieving a desired level of efflux inhibition.

### SUMMARY OF THE INVENTION

One aspect of the invention is based on the unexpected discovery that the efflux inhibition effect of TPGS varies with the molecular weight of the polyethylene glycol (PEG) portion of the molecule. In some embodiments, it has been found that over the PEG molecular weight range of 200-6,000 atomic mass units (amu), efflux inhibition reaches a maximum between about 750 and about 2500. Curve fitting in these embodiments suggest a maximum efflux inhibitory effect around 1,300 PEG MW.

In some embodiments, TPGS molecules having a PEG molecular weight of no more than about 600 exhibit a significant solubilizing effect when coadministered with lipophilic drugs, without exhibiting significant efflux inhibition. In some embodiments, TPGS molecules having a PEG molecular weight of at least about 3400 exhibit a significant solubilizing effect when coadministered with lipophilic drugs, without exhibiting significant efflux inhibition. Properties that provide useful solubilizing agents without efflux inhibition characteristics will allow formulators a choice to simultaneously obtain solubilizing effects with efflux inhibition of a desired degree, or a product having solubilizing effects but lacking substantial efflux inhibition.

### BRIEF DESCRIPTION OF THE DRAWINGS

FIG. 1 depicts the molecular structure of TPGS in which n=the number of ethylene glycol monomers present in the PEG portion of the molecule. The numerical value in the TPGS designation refers to the molecular weight of the PEG from which it was made. Thus, TPGS 1000 contains a PEG side chain with an average molecular weight of 1000. This can be converted to the number of ethylene glycol monomers in the chain by subtracting 18 amu and dividing by 44. Thus, a PEG MW of 1000 average molecular weight is the product of condensation of approximately 22.3 ethylene glycol monomers, meaning that “n” is ˜22.

FIG. 2 depicts the dependence of the inhibitory effect on the length of the PEG chain. Weibull regression of relative secretory transport decrease and relative absorptive transport increase (mean±SD, n=18) where (F(x)=1−exp [−(x/b)^{c}], 0<x) have r² values of ˜0.94 and ˜0.91, respectively. Using these calculations, the predicted optimal PEG chain length for maximal absorptive and secretory RHO transport reside between 1581 (±209) and 1182 (±476) Da, respectively.

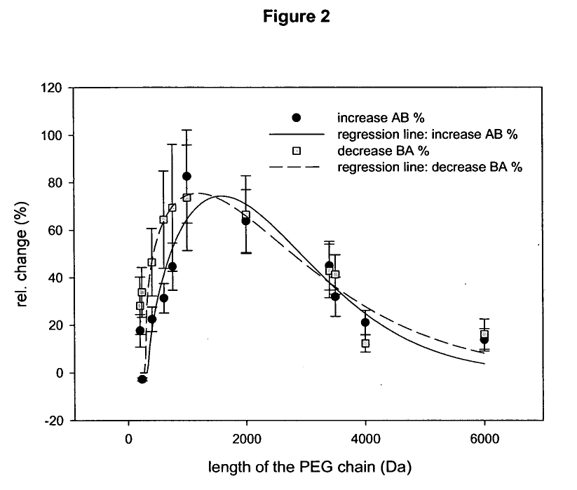

FIG. 3 depicts the dependence of permeability coefficient (in both the basolateral to apical and apical to basolateral directions) of Caco-2 monolayers to Rhodamine 123 in the presence of Vitamin E TPGS upon the molecular weight of the PEG portion of Vitamin E TPGS. “Rho 123” on the x-axis refers to Rhodamine 123 alone as a negative control. For other data points, the x-axis indicates the molecular weight of the PEG. “3400” is actually data for PEG MW 3350, that has been rounded up for purposes of the figure. Y-axis indicates the permeability coefficient in each direction. The darker shaded bars indicate apical to basolateral direction, while the lighter bars indicate basolateral to apical direction. Bar height indicates mean values (mean±SD, n=18), with vertical lines extending from the top of the bars indicating standard deviation. Bars marked with * are significantly different from negative control (p<0.05) and ** are very significantly different (P<0.001). Data were determined using the Inhibition Protocol. Portions of these data reflect some of the data in Examples 2-10.

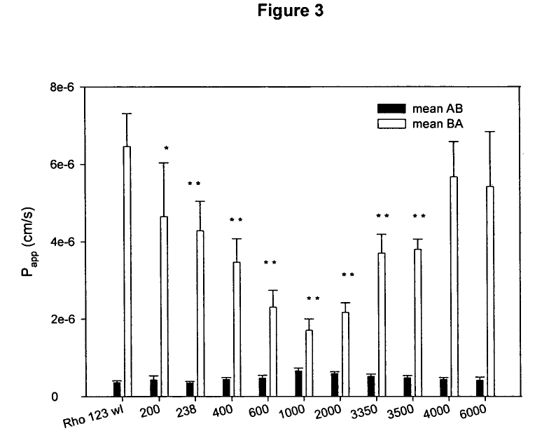

FIG. 4 depicts the Caco-2 monolayer permeability (apical to basolateral direction only) of Rhodamine 123 in the presence of Vitamin E TPGS in which the PEG Chain has varying molecular weights. “Rho 123” on the x-axis refers to Rhodamine 123 alone as a negative control. For other data points, the x-axis indicates the molecular weight of the PEG. “3400” is actually data for PEG MW 3350, that has been rounded up for purposes of the figure. Y-axis indicates the permeability coefficient. Bar height indicates mean values (mean±SD, n=18) with vertical lines extending from the top of the bars indicating standard deviation. Bars marked with * are significantly different from negative control (p<0.05) and ** are very significantly different (P<0.001). Data were determined using the Inhibition Protocol. Portions of these data reflect some of the data in Examples 2-10.

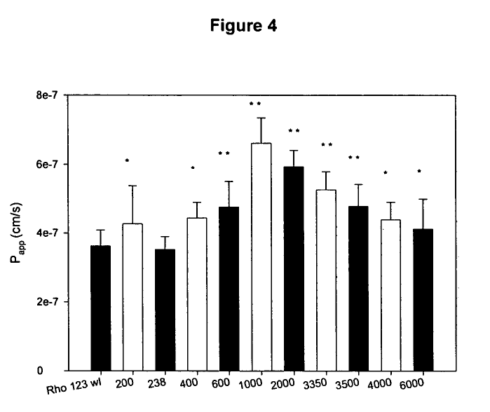

### DETAILED DESCRIPTION

Definitions

As used throughout this application, the term “lipophilic compounds” shall mean compounds having solubility in water that is in the “sparingly soluble” range, or lower. (Persons of ordinary skill in the art will understand that, for compounds that are “sparingly soluble in water,” the quantity of water needed to dissolve one gram of the compound will be in the range beginning at about 30 mL and ending at about 100 mL. Compounds having solubility lower than “sparingly soluble” in water will require greater volumes of water to dissolve the compounds).

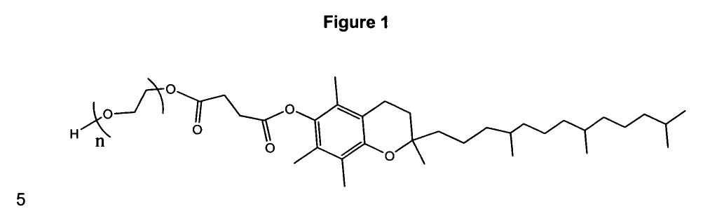

The term “TPGS,” “TPGS compound.” or “TPGS analog” shall refer to any compound depicted by FIG. 1.

As used throughout this application, the terms “effectively solubilizing” a compound or having a “solubilizing effect” on such compound shall mean having the effect of increasing the solubility in water of the compound at least about two-fold (i.e., reducing by at least about half the amount of water required to dissolve one gram of the compound).

As used throughout this application, the term “compound for pharmaceutical use” refers to any substance which, when administered to a human or animal under conditions effective to cause absorption to the bloodstream, or into target cells, tissues, or organs, causes a therapeutic or prophylactic effect. Examples of pharmaceuticals include, but are not limited to, anesthetics, hypnotics, sedatives and sleep inducers, antipsychotics, antidepressants, antiallergics, antianginals, antiarthritics, antiasthmatics, antidiabetics, antidiarrheal drugs, anticonvulsants, antigout drugs, antihistamines, antipruritics, emetics, antiemetics, antispasmondics, appetite suppressants, neuroactive substances, neurotransmitter agonists, antagonists, receptor blockers and reuptake modulators, beta-adrenergic blockers, calcium channel blockers, disulfarim and disulfarim-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, and monoamine oxidase inhibitors.

As used throughout this application, the term “lipophilic compound for pharmaceutical use” refers to a lipophilic compound that is also a compound for pharmaceutical use. Examples of lipophilic compounds for pharmaceutical use include, but are not limited to, itraconazole, astemizole, saquinavir, amprenavir, paclitaxel, docetaxel, doxorubicin, ibuprofen, posaconazole, tacrolimus, danazol, estrogen, lopinavir, tamoxifen, nevirapine, efavirenz, delaviridine, nelfinavir, raloxifene, erythromycin, carbamazepine, ketoconazole, indinavir, progesterone, ritonavir, etc.

As used throughout this application, the term “pharmaceutically effective amount of a lipophilic compound for pharmaceutical use” shall mean an amount of that compound that exhibits the intended pharmaceutical, prophylactic or therapeutic effect when administered.

As used throughout this application, the term “increasing bioavailability” or “increased bioavailability” of one or more compound(s) administered shall mean, in reference to the effect of administering a TPGS analog, that the TPGS analog results in an increase in the portion of the dose of the compound(s) administered that reaches one or more targeted systemic fluids, organs, tissues or cells as compared to administration without the TPGS analog. Increased bioavailability can include any mechanism that that has a desired effect on cellular efflux, cellular influx, or clearance. “Clearance” includes any type of elimination of one or more compounds from cells, blood, plasma, tissues or organs (e.g. intestinal clearance, hepatic clearance, renal clearance, and pulmonary clearance each describe elimination of compounds from the blood). Clearance may be described via the observed differences of renal excretion and elimination by all other processes including influx and efflux mechanisms (e.g. gastrointestinal clearance, excretory clearance, biliary clearance and enterohepatic cycling, metabolic clearance). Examples of systemic fluids include, but are not limited to: blood; cerebrospinal fluid; lymph; and any other tissue fluids (including increased amounts in tissues that are bathed by such fluids, such as the brain, tissue of one or more visceral organs, connective tissue, muscle, fat, or one or more tissues in the skin). In some embodiments, the increase is systemic, as in the case of an increase measurable anywhere in the blood. In some embodiments, the increase is more localized, as is the case with some embodiments involving topical administration in which the increase is measured only in areas near the administration. An increase in portion of the dosage that reaches a fluid or tissue measurable by any reliable means is within this definition, including but not limited to increases identified by measuring the total systemic drug concentration over time after administration. In some embodiments, concentrations are determined by measuring the tissue or fluids themselves, or by measuring fractions thereof (for example, without limitation, serum or plasma in the case of blood). In some embodiments, increases for compounds that are excreted metabolized and/or un-metabolized in urine are determined by measuring levels of compounds or metabolites of the compounds in urine and will reflect an increase in systemic concentrations. In some embodiments an increase in compound bioavailability is defined as an increase in the Area Under the Curve (AUC). AUC is an integrated measure of systemic compound concentrations over time in units of mass-time/volume and is measured from the time compound is administered (time zero) to infinity (when no compound(s) remaining in the body can be measured). Information regarding monitoring substances are known to persons of ordinary skill in the art and may be found in references such as M. Rowland and T. N. Tozer, Clinical Pharmacokinetics Concepts and Applications (third Ed., 1995), Lippincoft Willams and Wilkins, Philadelphia.

As used throughout this application, Pₐₚₚ refers to apparent permeability coefficient as defined in the Inhibition Protocol set forth herein. The term PₐₚₚBA refers to the permeability coefficient in the basolateral to apical direction determined using the Inhibition Protocol set forth herein. The term PₐₚₚAB refers to the permeability coefficient in the apical to basolateral direction determined using the Inhibition Protocol set forth herein.

As used throughout this application, the terms “compound for use as an efflux inhibitor” shall mean a compound that reduces PₐₚₚBA to no more than about 50% of the PₐₚₚBA observed in the absence of the compound as determined using the Inhibition Protocol set forth in this application. Thus, a “compound not for use as an efflux inhibitor” shall mean a compound that does not reduce PₐₚₚBA or that reduces PₐₚₚBA to an amount greater than 50% of PₐₚₚBA observed in the absence of the compound, as determined using the Inhibition Protocol. In some embodiments, the compound is a compound that reduces the efflux of drugs and other substances out of a cell, into the gut, or organ (brain, liver, kidney, etc.) due to any mechanism, including, but not limited to the action of enzymes or transporter proteins such as P-glycoprotein (P-gp), Breast Cancer Resistance Protein (BCRP), multi-drug resistant-associated proteins (MRP's), cytochrome P450's, UDP-glucuronosyltransferases and sulfotransferases, etc., as demonstrated using the Inhibition Protocol set forth herein. In some embodiments, the compound is one that causes increased bioavailability as defined above.

As used throughout this application, the term molecular weight, including the abbreviation MW, shall refer, in connection with a single molecule, to the molecular weight of that molecule. With respect to a polydisperse preparation containing polymer molecules of differing molecular weights, molecular weight shall refer to weight-average molecular weight (M_{W}).

TPGS and TPGS Compositions

The invention includes TPGS and compositions comprising TPGS. The TPGS contains a PEG that has a selected molecular weight or is within a selected range of molecular weights. The molecular weight is selected to provide a TPGS having a desired degree of efflux inhibition, or lack thereof. In some embodiments, the polyethylene glycol molecular weight is less than or equal to about 900. In some embodiments, the polyethylene glycol molecular weight is less than or equal to about 800. In some embodiments, the polyethylene glycol molecular weight is less than or equal to about 700. In some embodiments, the polyethylene glycol molecular weight is less than or equal to about 600. In some embodiments, the polyethylene glycol molecular weight is less than or equal to about 500. In some embodiments, the polyethylene glycol molecular weight is less than or equal to about 400. In some embodiments, the polyethylene glycol molecular weight is less than or equal to about 300. In some embodiments, the polyethylene glycol molecular weight is less than or equal to about 200. In some embodiments, the polyethylene glycol molecular weight is greater than or equal to about 1500. In some embodiments, the polyethylene glycol molecular weight is greater than or equal to about 1600. In some embodiments, the polyethylene glycol molecular weight is greater than or equal to about 1700. In some embodiments, the polyethylene glycol molecular weight is greater than or equal to about 1800. In some embodiments, the polyethylene glycol molecular weight is greater than or equal to about 1900. In some embodiments, the polyethylene glycol molecular weight is less than or equal to about 2000. In some embodiments, the polyethylene glycol molecular weight is greater than or equal to about 2100. In some embodiments, the polyethylene glycol molecular weight is greater than or equal to about 2200. In some embodiments, the polyethylene glycol molecular weight is greater than or equal to about 2300. In some embodiments, the polyethylene glycol molecular weight is greater than or equal to about 2400. In some embodiments, the polyethylene glycol molecular weight is greater than or equal to about 2500. In some embodiments, the polyethylene glycol molecular weight is greater than or equal to about 2600. In some embodiments, the polyethylene glycol molecular weight is less than or equal to about 3000. In some embodiments the polyethylene glycol molecular weight is greater than or equal to 3350. In some embodiments, the polyethylene glycol molecular weight is greater than or equal to about 3500. In some embodiments, the polyethylene glycol molecular weight is less than or equal to about 4000. In some embodiments, the polyethylene glycol molecular weight is greater than or equal to about 4500. In some embodiments, the polyethylene glycol molecular weight is less than or equal to about 5000. In some embodiments, the polyethylene glycol molecular weight is greater than or equal to about 5500. In some embodiments, the polyethylene glycol molecular weight is less than or equal to about 6000. In some embodiments, the polyethylene glycol molecular weight is less than or equal to about 7000. In some embodiments, the polyethylene glycol molecular weight is less than or equal to about 8000. In some embodiments, the polyethylene glycol molecular weight is less than or equal to about 9000. In some embodiments, the polyethylene glycol molecular weight is less than or equal to about 10000. In some embodiments, the polyethylene glycol molecular weight is less than or equal to about 11000. In some embodiments, the polyethylene glycol molecular weight is less than or equal to about 12000. In some embodiments, the polyethylene glycol molecular weight is less than or equal to about 13000. In some embodiments, the polyethylene glycol molecular weight is less than or equal to about 14000. In some embodiments, the polyethylene glycol molecular weight is less than or equal to about 15000. In some embodiments, the polyethylene glycol molecular weight is less than or equal to about 16000. In some embodiments, the polyethylene glycol molecular weight is less than or equal to about 17000. In some embodiments, the polyethylene glycol molecular weight is less than or equal to about 18000. In some embodiments, the polyethylene glycol molecular weight is less than or equal to about 19000. In some embodiments, the polyethylene glycol molecular weight is less than or equal to about 20000. In some embodiments, the polyethylene glycol molecular weight is less than or equal to about 22000. In some embodiments, the polyethylene glycol molecular weight is less than or equal to about 24000. In some embodiments, the polyethylene glycol molecular weight is less than or equal to about 26000. In some embodiments, the polyethylene glycol molecular weight is less than or equal to about 28000. In some embodiments, the polyethylene glycol molecular weight is less than or equal to about 30000. In some embodiments, the polyethylene glycol molecular weight is less than or equal to about 32000. In some embodiments, the polyethylene glycol molecular weight is less than or equal to about 34000. In some embodiments, the polyethylene glycol molecular weight is less than or equal to about 36000. In some embodiments, the polyethylene glycol molecular weight is less than or equal to about 38000. In some embodiments, the polyethylene glycol molecular weight is less than or equal to about 41000. Embodiments also exist in which the molecular weight of the polyethylene glycol is in specific ranges, for example 50-150, 100-200, 150-250, 200-300, 250-350, 300-400, 350-450, 400-500, 450-550, 500-600, 550-650, 600-700, 650-750, 700-800, 750-850, 800-900, 850-950, 1000-1100, 1050-1150, 1100-1200, 1150-1250, 1200-1300, 1250-1350, 1300-1400, 1350-1450, 1400-1500, 1450-1550, 1500-1600, 1550-1650, 1600-1700, 1650-1750, 1700-1800, 1750-1850, 1800-1900, 1850-1950, 1900-2000, 1950-2050, 2000-2100, 2050-2150, 2100-2200, 2150-2250, 2200-2300, 2250-2350, 2300-2400, 2350-2450, 2400-2500, 2450-2550, 2500-2600, 2550-2650, 2600-2700, 2650-2750, 2700-2800, 2750-2850, 2800-2900, 2850-2950, 2900-3000, 2950-3050, 3000-3100, 3050-3150, 3100-3200, 3150-3250, 3200-3300, 3250-3350, 3300-3400, 3350-3450, 3400-3500, 3450-3550, 3500-3600, 3550-3650, 3600-3700, 3650-3750, 3700-3800, 3750-3850, 3800-3900, 3850-3950, 3900-4000, 3950-4050, 4000-4100, 4050-4150, 4100-4200, 4150-4250, 4200-4300, 4250-4350, 4300-4400, 4350-4450, 4400-4500, 4450-4550, 4500-4600, 4550-4650, 4600-4700, 4650-4750, 4700-4800, 4750-4850, 4800-4900, 4850-4950, 4900-6000, 4950-6050, 5000-5100, 5050-5150, 5100-5200, 5150-5250, 5200-5300, 5250-5350, 5300-5400, 5350-5450, 5400-5500, 5450-5550, 5500-5600, 5550-5650, 5600-5700, 5650-5750, 5700-5800, 5750-5850, 5800-5900, 5850-5950, 5900-6000, 5950-6050, 6000-6100, 6050-6150, 6100-6200, 6150-6250, 6200-6300, 6250-6350, 6300-6400, 6350-6450, 6400-6500, 6450-6550, 6500-6600, 6550-6650, 6600-6700, 6650-6750, 6700-6800, 6750-6850, 6800-6900, 6850-6950, 6900-7000, 6950-7050, 7000-7100, 7050-7150, 7100-7200, 7150-7250, 7200-7300, 7250-7350, 7300-7400, 7350-7450, 7400-7500, 7450-7550, 7500-7600, 7550-7650, 7600-7700, 7650-7750, 7700-7800, 7750-7850, 7800-7900, 7850-7950, 7900-8000, 7950-8050, 8000-8100, 8050-8150, 8100-8200, 8150-8250, 8200-8300, 8250-8350, 8300-8400, 8350-8450, 8400-8500, 8450-8550, 8500-8600, 8550-8650, 8600-8700, 8650-8750, 8700-8800, 8750-8850, 8800-8900, 8850-8950, 8900-9000, 8950-9050, 9000-9100, 9050-9150, 9100-9200, 9150-9250, 9200-9300, 9250-9350, 9300-9400, 9350-9450, 9400-9500, 9450-9550, 9500-9600, 9550-9650, 9600-9700, 9650-9750, 9700-9800, 9750-9850, 9800-9900, 9850-9950, 9900-10000, 9950-10050, 10000-10100, 10050-10150, 10100-10200, 10150-10250, 10200-10300, 10250-10350, 10300-10400, 10350-10450, 10400-10500, 10450-10550, 10500-10600, 10550-10650, 10600-10700, 10650-10750, 10700-10800, 10750-10850, 10800-10900, 10850-10950, 10900-11000, 10950-11050, 11000-11100, 11050-11150, 11100-11200, 11150-11250, 11200-11300, 11250-11350, 11300-11400, 11350-11450, 11400-11500, 11450-11550, 11500-11600, 11550-11650, 11600-11700, 11650-11750, 11700-11800, 11750-11850, 11800-11900, 11850-11950, 11900-12000, 11950-12050, 12000-12100, 12050-12150, 12100-12200, 12150-12250, 12200-12300, 12250-12350, 12300-12400, 12350-12450, 12400-12500, 12450-12550, 12500-12600, 12550-12650, 12600-12700, 12650-12750, 12700-12800, 12750-12850, 12800-12900, 12850-12950, 12900-13000, 12950-13050, 13000-13100, 13050-13150, 13100-13200, 13150-13250, 13200-13300, 13250-13350, 13300-13400, 13350-13450, 13400-13500, 13450-13550, 13500-13600, 13550-13650, 13600-13700, 13650-13750, 13700-13800, 13750-13850, 13800-13900, 13850-13950, 13900-14000, 13950-14050, 14000-14100, 14050-14150, 14100-14200, 14150-14250, 14200-14300, 14250-14350, 14300-14400, 14350-14450, 14400-14500, 14450-14550, 14500-14600, 14550-14650, 14600-14700, 14650-14750, 14700-14800, 14750-14850, 14800-14900, 14850-14950, 14900-14000, 15950-15050, 15000-15100, 15050-15150, 15100-15200, 15150-15250, 15200-15300, 15250-15350, 15300-15400, 15350-15450, 15400-15500, 15450-15550, 15500-15600, 15550-15650, 15600-15700, 15650-15750, 15700-15800, 15750-15850, 15800-15900, 15850-15950, 15900-14000, 16950-16050, 17000-17100, 17050-17150, 17100-17200, 17150-17250, 17200-17300, 17250-17350, 17300-17400, 17350-17450, 17400-17500, 17450-17550, 17500-17600, 17550-17650, 17600-17700, 17650-17750, 17700-17800, 17750-17850, 17800-17900, 17850-17950, 17900-14000, 17950-18050, 18000-18100, 18050-18150, 18100-18200, 18150-18250, 18200-18300, 18250-18350, 18300-18400, 18350-18450, 18400-18500, 18450-18550, 18500-18600, 18550-18650, 18600-18700, 18650-18750, 18700-18800, 18750-18850, 18800-18900, 18850-18950, 18900-14000, 18950-19050, 19000-19100, 19050-19150, 19100-19200, 19150-19250, 19200-19300, 19250-19350, 19300-19400, 19350-19450, 19400-19500, 19450-19550, 19500-19600, 19550-19650, 19600-19700, 19650-19750, 19700-19800, 19750-19850, 19800-19900, 19850-19950, 19900-20000, 19950-20050, 20000-20100, 20050-20150, 20100-20200, 20150-20250, 20200-20300, 20250-20350, 20300-20400, 20350-20450, 20400-20500, 20450-20550, 20500-20600, 20550-20650, 20600-20700, 20650-20750, 20700-20800, 20750-20850, 20800-20900, 20850-20950, 20900-21000, 20950-21050, 21000-21100, 21050-21150, 21100-21200, 21150-21250, 21200-21300, 21250-21350, 21300-21400, 21350-21450, 21400-21500, 21450-21550, 21500-21600, 21550-21650, 21600-21700, 21650-21750, 21700-21800, 21750-21850, 21800-21900, 21850-21950, 21900-22000, 21950-22050, 22000-22100, 22050-22150, 22100-22200, 22150-22250, 22200-22300, 22250-22350, 22300-22400, 22350-22450, 22400-22500, 22450-22550, 22500-22600, 22550-22650, 22600-22700, 22650-22750, 22700-22800, 22750-22850, 22800-22900, 22850-22950, 22900-23000, 22950-23050, 23000-23100, 23050-23150, 23100-23200, 23150-23250, 23200-23300, 23250-23350, 23300-23400, 23350-23450, 23400-23500, 23450-23550, 23500-23600, 23550-23650, 23600-23700, 23650-23750, 23700-23800, 23750-23850, 23800-23900, 23850-23500, 23900-24000, 23950-24050, 24000-24100, 24050-24150, 24100-24200, 24150-24250, 24200-24300, 24250-24350, 24300-24400, 24350-24450, 24400-24500, 24450-24550, 24500-24600, 24550-24650, 24600-24700, 24650-24750, 24700-24800, 24750-24850, 24800-24900, 24850-24950, 24900-25000, 24950-25050, 25000-25100, 25050-25150, 25100-25200, 25150-25250, 25200-25300, 25250-25350, 25300-25400, 25350-25450, 25400-25500, 25450-25550, 25500-25600, 25550-25650, 25600-25700, 25650-25750, 25700-25800, 25750-25850, 25800-25900, 25850-25950, 25900-26000, 25950-26050, 26000-26100, 26050-26150, 26100-26200, 26150-26250, 26200-26300, 26250-26350, 26300-26400, 26350-26450, 26400-26500, 26450-26550, 26500-26600, 26550-26650, 26600-26700, 26650-26750, 26700-26800, 26750-26850, 26800-26900, 26850-26950, 26900-27000, 26950-27050, 27000-27100, 27050-27150, 27100-27200, 27150-27250, 27200-27300, 27250-27350, 27300-27400, 27350-27450, 27400-27500, 27450-27550, 27500-27600, 27550-27650, 27600-27700, 27650-27750, 27700-27800, 27750-27850, 27800-27900, 27850-27950, 27900-28000, 27950-28050, 28000-28100, 28050-28150, 28100-28200, 28150-28250, 28200-28300, 28250-28350, 28300-28400, 28350-28450, 28400-28500, 28450-28550, 28500-28600, 28550-28650, 28600-28700, 28650-28750, 28700-28800, 28750-28850, 28800-28900, 28850-28950, 28900-29000, 28950-29050, 29000-29100, 29050-29150, 29100-29200, 29150-29250, 29200-29300, 29250-29350, 29300-29400, 29350-29450, 29400-29500, 29450-29550, 29500-29600, 29550-29650, 29600-29700, 29650-29750, 29700-29800, 29750-29850, 29800-29900, 29850-29950, 29900-30000, 29950-30050, 30000-30100, 30050-30150, 30100-30200, 30150-30250, 30200-30300, 30250-30350, 30300-30400, 30350-30450, 30400-30500, 30450-30550, 30500-30600, 30550-30650, 30600-30700, 30650-30750, 30700-30800, 30750-30850, 30800-30900, 30850-30950, 30900-31000, 30950-31050, 31000-31100, 31050-31150, 31100-31200, 31150-31250, 31200-31300, 31250-31350, 31300-31400, 31350-31450, 31400-31500, 31450-31550, 31500-31600, 31550-31650, 31600-31700, 31650-31750, 31700-31800, 31750-31850, 31800-31900, 31850-31950, 31900-32000, 31950-32050, 32000-32100, 32050-32150, 32100-32200, 32150-32250, 32200-32300, 32250-32350, 32300-32400, 32350-32450, 32400-32500, 32450-32550, 32500-32600, 32550-32650, 32600-32700, 32650-32750, 32700-32800, 32750-32850, 32800-32900, 32850-32950, 32900-33000, 32950-33050, 33000-33100, 33050-33150, 33100-33200, 33150-33250, 33200-33300, 33250-33350, 33300-33400, 33350-33450, 33400-33500, 33450-33550, 33500-33600, 33550-33650, 33600-33700, 33650-33750, 33700-33800, 33750-33850, 33800-33900, 33850-33950, 33900-34000, 33950-34050, 34000-34100, 34050-34150, 34100-34200, 34150-34250, 34200-34300, 34250-34350, 34300-34400, 34350-34450, 34400-34500, 34450-34550, 34500-34600, 34550-34650, 34600-34700, 34650-34750, 34700-34800, 34750-34850, 34800-34900, 34850-34950, 34900-35000, 34950-35050, 35000-35100, 35050-35150, 35100-35200, 35150-35250, 35200-35300, 35250-35350, 35300-35400, 35350-35450, 35400-35500, 35450-35550, 35500-35600, 35550-35650, 35600-35700, 35650-35750, 35700-35800, 35750-35850, 35800-35900, 35850-35950, 35900-36000, 35950-36050, 36000-36100, 36050-36150, 36100-36200, 36150-36250, 36200-36300, 36250-36350, 36300-36400, 36350-36450, 36400-36500, 36450-36550, 36500-36600, 36550-36650, 36600-36700, 36650-36750, 36700-36800, 36750-36850, 36800-36900, 36850-36950, 36900-37000, 36950-37050, 37000-37100, 37050-37150, 37100-37200, 37150-37250, 37200-37300, 37250-37350, 37300-37400, 37350-37450, 37400-37500, 37450-37550, 37500-37600, 37550-37650, 37600-37700, 37650-37750, 37700-37800, 37750-37850, 37800-37900, 37850-37950, 37900-38000, 37950-38050, 38000-38100, 38050-38150, 38100-38200, 38150-38250, 38200-38300, 38250-38350, 38300-38400, 38350-38450, 38400-38500, 38450-38550, 38500-38600, 38550-38650, 38600-38700, 38650-38750, 38700-38800, 38750-38850, 38800-38900, 38850-38950, 38900-39000, 38950-39050, 39000-39100, 39050-39150, 39100-39200, 39150-39250, 39200-39300, 39250-39350, 39300-39400, 39350-39450, 39400-39500, 39450-39550, 39500-39600, 39550-39650, 39600-39700, 39650-39750, 39700-39800, 39750-39850, 39800-39900, 39850-39950, 39900-40000, 39950-40050, 40000-40100, 40050-40150, 40100-40200, 40150-40250, 40200-40300, 40250-40350, 40300-40400, 40350-40450, 40400-40500, 40450-40550, 40500-40600, 40550-40650, 40600-40700, 40650-40750, 40700-40800, 40750-40850, 40800-40900, 40850-40950, 40900-41000, 40950-41050. Embodiments also exist in which PEG is in a larger range made by combining two or more of any of the foregoing ranges. The invention includes single PEG molecules as well as groups of PEG molecules such as polydisperse pluralities of PEG molecules. In embodiments involving a polydisperse plurality of PEG molecules, “molecular weight” for such pluralities refers to M_{W}.

TPGS has the ability to form micelles in water, thereby helping to solubilize lipophilic compounds in water. For amphiphilic molecules such as TPGS, critical micellar concentration (CMC) in water is a good indicator of the compound's capability to effectively solubilize lipophilic compounds since it indicates how readily the compound will form micelles in water. In some embodiments, the TPGS contains a PEG of a molecular weight differing from that of TPGS 1000 but has a CMC that is no more than ten times the CMC of TPGS 1000. Information regarding CMC's are known to persons of ordinary skill in the art and may be found in references such as P. W. Atkins, Physical Chemistry (Fourth Edition, 1990), W.H. Freeman and Company, New York. In some embodiments, the TPGS contains a PEG of a molecular weight differing from that of TPGS 1000 but has a CMC that is no more than five times the CMC of TPGS 1000. In some embodiments, the TPGS contains a PEG of a molecular weight differing from that of TPGS 1000 but has a CMC that is no more than double the CMC of TPGS 1000. In some embodiments, the TPGS contains a PEG of a molecular weight differing from that of TPGS 1000 but has a CMC that is no more than 150% of the CMC of TPGS 1000. In some embodiments, the TPGS contains a PEG of a molecular weight differing from that of TPGS 1000 but has a CMC that is no more than 125% of the CMC of TPGS 1000. In some embodiments, the CMC of TPGS is 0.02±0.02 Wt % at all PEG molecular weights between 200 and 6000. In some embodiments, the TPGS contains a PEG of a molecular weight differing from that of TPGS 1000 but has a CMC that is the same as the CMC of TPGS 1000. The invention includes embodiments having any of the foregoing CMCs at each of the ranges of molecular weights (and combinations ranges of molecular weights) disclosed herein.

In some embodiments the TPGS is a “compound for use as an efflux inhibitor” as defined herein. In some embodiments the TPGS is a “compound not for use as an efflux inhibitor” as defined herein. In some embodiments in which the TPGS is a compound not for use as an efflux inhibitor, PₐₚₚBA of the TPGS is greater than about 60% of PₐₚₚBA in the absence of the TPGS as determined using the Inhibition Protocol. In some embodiments of compounds not for use as an efflux inhibitor, the PₐₚₚBA of the TPGS is greater than about 65% of PₐₚₚBA of in the absence of the TPGS as determined using the Inhibition Protocol. In some embodiments of compounds not for use as an efflux inhibitor, the PₐₚₚBA of the TPGS is greater than about 70% of PₐₚₚBA in the absence of the TPGS as determined using the Inhibition Protocol. In some embodiments of compounds not for use as an efflux inhibitor, the PₐₚₚBA of the TPGS is greater than about 75% of PₐₚₚBA of in the absence of the TPGS as determined using the Inhibition Protocol. In some embodiments of compounds not for use as an efflux inhibitor, the PₐₚₚBA of the TPGS is greater than about 80% of PₐₚₚBA in the absence of the TPGS as determined using the Inhibition Protocol. In some embodiments of compounds not for use as an efflux inhibitor, the PₐₚₚBA of the TPGS is greater than about 85% of PₐₚₚBA in the absence of the TPGS as determined using the Inhibition Protocol. In some embodiments of compounds not for use as an efflux inhibitor, the PₐₚₚBA of the TPGS is greater than about 90% of PₐₚₚBA in the absence of the TPGS as determined using the Inhibition Protocol. In some embodiments of compounds not for use as an efflux inhibitor, the PₐₚₚBA of the TPGS is greater than about 95% of PₐₚₚBA in the absence of the TPGS as determined using the Inhibition Protocol.

The invention also includes compositions that contain a TPGS of the present invention. Embodiments of such compositions exist involving all TPGS compounds described in this application as well as all combinations of such compounds. In some embodiments, the composition contains one or more lipophilic compounds along with a TPGS of the present invention. In some embodiments, the lipophilic compound is a lipophilic compound for pharmaceutical use. In some embodiments, the compositions contain a pharmaceutically effective amount of a lipophilic compound for pharmaceutical use. The TPGS in some embodiments is present above its CMC and thus increases the solubility of the lipophilic compound in water. In some embodiments, the TPGS is a compound that effectively solubilizes the lipophilic compound in water.

The invention further includes compositions that contain a plurality of TPGS molecules wherein the TPGS molecules are all within a single MW range disclosed above or within any combination or plurality of MW ranges.

In some embodiments, the compositions of the present invention contain one or more additional desirable components or compounds. Any desirable compounds are used. Examples include, but are not limited to, additional active pharmaceutical ingredients as well as excipients, diluents, and carriers such as fillers and extenders (e.g., starch, sugars, mannitol, and silicic derivatives); binding agents (e.g., carboxymethyl cellulose and other cellulose derivatives, alginates, gelatin, and polyvinyl-pyrrolidone); moisturizing agents (e.g., glycerol); disintegrating agents (e.g., calcium carbonate and sodium bicarbonate); agents for retarding dissolution (e.g., paraffin); resorption accelerators (e.g., quaternary ammonium compounds); surface active agents (e.g., cetyl alcohol, glycerol monostearate); adsorptive carriers (e.g., kaolin and bentonite); emulsifiers; preservatives; sweeteners; stabilizers; antioxidants; buffers; bacteriostats; coloring agents; perfuming agents; flavoring agents; lubricants (e.g., talc, calcium and magnesium stearate); solid polyethyl glycols; and mixtures thereof.

Examples of carriers include, without limitation, any liquids, liquid crystals, solids or semi-solids, such as water or saline, gels, creams, salves, solvents, diluents, fluid ointment bases, ointments, pastes, implants, liposomes, micelles, giant micelles, and the like, which are suitable for use in the compositions.

It should be understood that the ingredients particularly mentioned above are merely examples and that some embodiments of formulations comprising the compositions of the present invention include other suitable components and agents.

The invention further includes packages, vessels, or any other type of container that contain a TPGS of the present invention or any composition comprising a TPGS of the present invention. The package, vessel or container contains, is labeled with, or is otherwise accompanied by instructions to use the TPGS or TPGS composition to enhance or to increase solubility of one or more lipophilic compounds in water and indicates in any manner that the TPGS or TPGS composition has a specified degree of effect on efflux or otherwise causes a specified degree of increased bioavailability. Any degree of efflux inhibition or other increased bioavailability may be indicated. In some embodiments, the indication is that the TPGS or TPGS composiion does not inhibit efflux, has a diminished, limited, or insignificant inhibitory effect on efflux or increased bioavailability, or otherwise provides some indication regarding a lack of efflux inhibition or lack of increased bioavailability or a reduced degree of efflux inhibition or other increased bioavailability (for example, identifying that the efflux inhibition is no greater than a certain level).

Methods

The invention further includes various methods that use the TPGS and TPGS compositions described above. Any of the foregoing molecules and compositions (and combinations of such molecules and compositions) that are effective to produce a desired result can be used with each of such methods.

The compositions are administered in any form by any means. Examples of forms of administration include but are not limited to injections, solutions, creams, gels, implants, ointments, emulsions, suspensions, microspheres, powders, particles, microparticles, nanoparticles, liposomes, pastes, patches, capsules, suppositories, tablets, transdermal delivery devices, sprays, suppositories, aerosols, or other means familiar to one of ordinary skill in the art. In some embodiments, the compositions are combined with other components. Examples include but are not limited to coatings, depots, matrices for time release and osmotic pump components.

Examples of methods of administration include, but are not limited to, oral administration (e.g., ingestion, buccal or sublingual administration), anal or rectal administration, topical application, aerosol application, inhalation, intraperitoneal administration, intravenous administration, transdermal administration, intradermal administration, subdermal administration, intramuscular administration, intrauterine administration, vaginal administration, administration into a body cavity, surgical administration (for example, at the location of a tumor or internal injury), administration into the lumen or parenchyma of an organ, and parenteral administration.

In some embodiments, the compositions of the present invention are administered to persons or animals to provide substances in any dose range that will produce desired physiological or pharmacological results. Dosage will depend upon the substance or substances administered, the therapeutic endpoint desired, the desired effective concentration at the site of action or in a body fluid, and the type of administration. Information regarding appropriate doses of substances are known to persons of ordinary skill in the art and may be found in references such as L. S. Goodman and A. Gilman, eds, The Pharmacological Basis of Therapeutics, Macmillan Publishing, New York, and Katzung, Basic & Clinical Pharmacology, Appleton & Lang, Norwalk, Conn., (6th Ed. 1995).

The invention further includes any method of admixture or coadministration, including the above methods, in which the method further includes the step of identifying a desired degree (or lack thereof) of efflux inhibition on the part of the TPGS. In some embodiments, the method includes selecting from among several TPGS molecules (having different PEG molecular weights) that are compounds for use as an efflux inhibitor to identify the desired level of efflux inhibition. In some embodiments, the method includes selecting from among two or more TPGS molecules (having different PEG molecular weights) that are compounds not for use as an efflux inhibitor to identify the desired level of efflux inhibition. In some embodiments, the method includes selecting from among several TPGS molecules (having different PEG molecular weights) that are compounds for use as an efflux inhibitor as well as several TPGS molecules that are compounds not for use as an efflux inhibitor to identify the desired level of efflux inhibition. In some embodiments, the method includes selecting a mixture or other combination of a plurality of TPGS molecules having different PEG molecular weights to obtain the desired degree of efflux inhibition. Through this method, manipulation of the molecular weight or weights of the PEG portion of the TPGS allows fine control of the degree of efflux inhibition.

Inhibition Protocol (Caco-2 Monolayer Culture Protocol)

As used herein, the term “Inhibition Protocol” or “inhibition protocol” refers to the following test. The test is carried out using Caco-2 (C2BBe1 or HTB-37) monolayers which are known to be a good in vitro model for gastrointestinal epithelial cells. The following describes the inhibition protocol: Caco-2 cells, clone C2BBe1, from passages 48-92 are used. Cells are grown to ˜90% confluent in 75 cm² T-flasks with Dulbecco's modified Eagle's medium (DMEM) supplemented with 10% fetal bovine serum (FBS) and 1% non-essential amino-acids. Cells are grown at a temperature of ˜37° C. in an atmosphere of ˜85% relative humidity and ˜5% CO₂. Cells are seeded on top of Transwell® inserts (pore size 0.4 μm, 1.13 cm²) at a density of ˜60,000 cells/cm². Caco-2 monolayers are used ˜21-25 days after seeding. Transepithelial electrical resistance (TEER) are measured and monolayers only with a TEER>350 Ω*cm², with background subtracted, are used for transport studies.

Rhodamine 123 (RHO) transport is assessed in absorptive (apical to basolateral, Ap→BI) and secretory (BI→Ap) directions. Prior to the RHO transport experiments, the monolayers were pre-incubated (˜1 h) with the corresponding TPGS analog (˜33 μM) on both sides. Subsequently, at t=0 min, a solution of RHO (˜33 μM in Krebs Ringer Buffer (KRB) pH 7.4) is added to the donor compartment and pure HBSS (pH 7.4) to the receiver compartment, such that both sides contain TPGS analog (˜33 μM).

Monolayers are agitated using an orbital shaker at ˜100±20 rpm. Samples were taken after 30, 60, 120, 180, 240, and 300 min from the receiver compartment. After each sampling, an equal volume of fresh transport buffer (˜37° C.) was added to the receiver compartment. Experiments were performed over 3 passages, each directional transport experiment comprising a total of n=18. To ensure integrity of the monolayers, TEER values were measured on the day of the experiment and at the end of the experiment.

Flux was determined using receiver compartment RHO steady-state appearance rates (dQ/dt; μg/s). Apparent permeability (Pₐₚₚ) was calculated according to:
Pₐₚₚ=(dQ/dt)*(1/A)*(1/Cₒ)

where A (cm²) is the nominal surface area of the monolayer and C₀ (μg/mL) is the RHO concentration in the donor compartment at t=0. Relative change of Pₐₚₚ(cm/s)was calculated according to the equation:
rel. increase/decrease=|(1−Pₐₚₚ(TPGS)/Pₐₚₚ(RHO))*100|.

PₐₚₚBA/PₐₚₚAB (efflux ratio) is the ratio of PₐₚₚBA divided by PₐₚₚAB. Significance of difference in the Pₐₚₚ values were determined by one-way analysis of variances (ANOVA) followed by Neumann-Keuls-Student post-hoc tests.

This invention can be further illustrated by the following examples of preferred embodiments thereof, although it will be understood that these examples are included merely for purposes of illustration and are not intended to limit the scope of the invention unless otherwise specifically indicated.

### EXAMPLES

Examples 1(a) to (n) illustrate the ability of TPGS 400 to solubilize lipophilic drugs. These examples use the following commercially available products.

Vital Nutrients CoEnzyme Q10 Powder (Vital Nutrients/RHG & CO., Inc. Middletown, Conn. 06457 USA)

Ibuprofen, USP-25, Mesh: 5-10 micron (DASTECH International, Inc. 10 Cutter Mill Road, Great Neck, N.Y. 11021)

Lipoic Acid (Medical Research Institute, 444 DeHaro Street, Suite 209, San Francisco, Calif. 94107-2347)

Vitamin E TPGS 400 (Available from Eastman Chemical Company, Kingsport, Tenn.)

### Example 1(a)

TPGS 400/CoEnzyme Q10 (90:10)

Nine grams of Eastman Vitamin E TPGS 400, a water-dispersible form of vitamin E, were weighed into a Pyrex Media bottle. One gram of CoEnzyme Q10 powder, a dietary supplement, was then added to the bottle. The bottle was sealed then placed in an oven. The oven temperature was set at 75 degrees Celsius. After six hours the sample was removed and mixed thoroughly using a vortexer. The sample was returned to the oven and after eighteen hours the oven was turned off. The sample was allowed to cool to room temperature then removed. The blend was free flowing and dark red in appearance. After one week the sample remained free flowing.

### Example 1(b)

TPGS 400/CoEnzyme Q10 (80:20)

Eight grams of Eastman Vitamin E TPGS 400, a water-dispersible form of vitamin E, were weighed into a Pyrex Media bottle. Two grams of CoEnzyme Q10 powder, a dietary supplement, were then added to the bottle. The bottle was sealed then placed in an oven. The oven temperature was set at 75 degrees Celsius. After six hours the sample was removed and mixed thoroughly using a vortexer. The sample was returned to the oven and after eighteen hours the oven was turned off. The sample was allowed to cool to room temperature then removed. The blend was free flowing and dark red in appearance. After three days the sample began to crystallize.

### Example 1(c)

TPGS 400/CoEnzyme Q10 (70:30)

Seven grams of Eastman Vitamin E TPGS 400, a water-dispersible form of vitamin E, were weighed into a Pyrex Media bottle. Three grams of CoEnzyme Q10 powder, a dietary supplement, were then added to the bottle. The bottle was sealed then placed in an oven. The oven temperature was set at 75 degrees Celsius. After six hours the sample was removed and mixed thoroughly using a vortexer. The sample was returned to the oven and after eighteen hours the oven was turned off. The sample was allowed to cool to room temperature then removed. The blend was free flowing and dark red in appearance. After three days the sample began to crystallize.

### Example 1(d)

TPGS 400/CoEnzyme Q10 (60:40)

Six grams of Eastman Vitamin E TPGS 400, a water-dispersible form of vitamin E, were weighed into a Pyrex Media bottle. Four grams of CoEnzyme Q10 powder, a dietary supplement, were then added to the bottle. The bottle was sealed then placed in an oven. The oven temperature was set at 75 degrees Celsius. After six hours the sample was removed and mixed thoroughly using a vortexer. The sample was returned to the oven and after eighteen hours the oven was turned off. The sample was allowed to cool to room temperature then removed. The blend was free flowing and dark red in appearance. After three days the sample began to crystallize.

### Example 1(e)

TPGS 400/CoEnzyme Q10 (50:50)

Five grams of Eastman Vitamin E TPGS 400, a water-dispersible form of vitamin E, were weighed into a Pyrex Media bottle. Five grams of CoEnzyme Q10 powder, a dietary supplement, were then added to the bottle. The bottle was sealed then placed in an oven. The oven temperature was set at 75 degrees Celsius. After six hours the sample was removed and mixed thoroughly using a vortexer. The sample was returned to the oven and after eighteen hours the oven was turned off. The sample was allowed to cool to room temperature then removed. The blend was free flowing and dark red in appearance. After three days the sample began to crystallize.

### Example 1(f)

TPGS 400/CoEnzyme Q10 (10:90)

One gram of Eastman Vitamin E TPGS 400, a water-dispersible form of vitamin E, was weighed into a Pyrex Media bottle. Nine grams of CoEnzyme Q10 powder, a dietary supplement, were then added to the bottle. The bottle was sealed then placed in an oven. The oven temperature was set at 75 degrees Celsius. After six hours the sample was removed and mixed thoroughly using a vortexer. The sample was returned to the oven and after eighteen hours the oven was turned off. The sample was allowed to cool to room temperature then removed. The blend was free flowing and dark red in appearance. After three days the sample began to crystallize.

### Example 1(g)

TPGS 400/CoEnzyme Q10 (90:10) In Water

Ninety grams of Millipore water were added to a Pyrex beaker. The beaker was placed in a heating mantle, and the water was heated to 65 degrees Celsius. Ten grams of a TPGS 400/CoEnzyme Q10 blend (90:10) were placed in an oven and heated to 65 degrees Celsius. The blend was added to the water with mixing. The heat source was turned off, and the sample was allowed to cool to room temperature with continued mixing. The dispersion is stable after one week.

### Example 1(h)

TPGS 400/Ibuprofen (90:10)

Nine grams of Eastman Vitamin E TPGS 400, a water-dispersible form of vitamin E, were weighed into a Pyrex Media bottle. One gram of ibuprofen, a non-steroidal anti-inflammatory, was then added to the bottle. The bottle was sealed then placed in an oven. The oven temperature was set at 75 degrees Celsius. After six hours the sample was removed and mixed thoroughly using a vortexer. The sample was returned to the oven and after eighteen hours the oven was turned off. The sample was allowed to cool to room temperature then removed. The blend was free flowing, clear and yellow in appearance. After one week the sample remained free flowing.

### Example 1(i)

TPGS 400/Ibuprofen (80:20)

Eight grams of Eastman Vitamin E TPGS 400, a water-dispersible form of vitamin E, were weighed into a Pyrex Media bottle. Two grams of ibuprofen, a non-steroidal anti-inflammatory, were then added to the bottle. The bottle was sealed then placed in an oven. The oven temperature was set at 75 degrees Celsius. After six hours the sample was removed and mixed thoroughly using a vortexer. The sample was returned to the oven and after eighteen hours the oven was turned off. The sample was allowed to cool to room temperature then removed. The blend was free flowing, clear and yellow in appearance. After one week the sample remained free flowing.

### Example 1(j)

TPGS 400/Ibuprofen (70:30)

Seven grams of Eastman Vitamin E TPGS 400, a water-dispersible form of vitamin E, were weighed into a Pyrex Media bottle. Three grams of ibuprofen, a non-steroidal anti-inflammatory, were then added to the bottle. The bottle was sealed then placed in an oven. The oven temperature was set at 75 degrees Celsius. After six hours the sample was removed and mixed thoroughly using a vortexer. The sample was returned to the oven and after eighteen hours the oven was turned off. The sample was allowed to cool to room temperature then removed. The blend was free flowing, clear and yellow in appearance. After one week the sample remained free flowing.

### Example 1(k)

TPGS 400/Ibuprofen (60:40)

Six grams of Eastman Vitamin E TPGS 400, a water-dispersible form of vitamin E, were weighed into a Pyrex Media bottle. Four grams of ibuprofen, a non-steroidal anti-inflammatory, were then added to the bottle. The bottle was sealed then placed in an oven. The oven temperature was set at 75 degrees Celsius. After six hours the sample was removed and mixed thoroughly using a vortexer. The sample was returned to the oven and after eighteen hours the oven was turned off. The sample was allowed to cool to room temperature then removed. The blend was free flowing, clear and yellow in appearance. After one week the sample began to crystallize.

### Example 1(l)

TPGS 400/Ibuprofen (50:50)

Five grams of Eastman Vitamin E TPGS 400, a water-dispersible form of vitamin E, were weighed into a Pyrex Media bottle. Five grams of ibuprofen, a non-steroidal anti-inflammatory, were then added to the bottle. The bottle was sealed then placed in an oven. The oven temperature was set at 75 degrees Celsius. After six hours the sample was removed and mixed thoroughly using a vortexer. The sample was returned to the oven and after eighteen hours the oven was turned off. The sample was allowed to cool to room temperature then removed. The blend was free flowing, clear and yellow in appearance. After one week the sample began to crystallize.

### Example 1(m)

TPGS 400/Ibuprofen (90:10) In Water

Ninety grams of Millipore water were added to a Pyrex beaker. The beaker was placed in a heating mantle, and the water was heated to 80 degrees Celsius. Ten grams of a TPGS 400/Ibuprofen blend (90:10) were placed in an oven and heated to 80 degrees Celsius. The blend was added to the water with mixing. The heat source was turned off, and the sample was allowed to cool to room temperature with continued mixing. The dispersion is stable after one week.

### Example 1(n)

TPGS 400/Lipoic Acid (90:10)

Nine grams of Eastman Vitamin E TPGS 400, a water-dispersible form of vitamin E, were weighed into a Pyrex Media bottle. One gram of Lipoic Acid, a dietary supplement, was then added to the bottle. The bottle was sealed then placed in an oven. The oven temperature was set at 75 degrees Celsius. After six hours the sample was removed and mixed thoroughly using a vortexer. The sample was returned to the oven and after eighteen hours the oven was turned off. The sample was allowed to cool to room temperature then removed. The blend was free flowing, cloudy and yellow in appearance. After one week the sample remained free flowing.

### Example 2

### Rhodamine 123 Efflux in Caco-2 Monolayers in the Presence of TPGS 1000

The Inhibition Protocol was performed with TPGS 1000.

| TPGS | | | | | |
|---|---|---|---|---|---|
| 1000 | Pₐₚₚ, A to | Pₐₚₚ, B to | Ratio, | % | % |
| Conc., | B × 10⁻⁷ | A × 10⁻⁷ | Pₐₚₚ(B − A)/ | Increase, | Decrease, |
| μmolar | cm/s | cm/s | Pₐₚₚ(A − B) | A to B | B to A |
| None | 3.62 | 64.7 | 17.9 | NA | NA |
| (Control) | | | | | |
| 33 | 6.62 | 17.1 | 2.58 | 82.5 | 73.6 |

This example shows that TPGS 1000 effectively inhibited efflux transport of Rhodamine 123 in Caco-2 monolayers. Rhodamine 123 is known to be affected by efflux transport in normal Caco-2 cells.

### Example 3

### Rhodamine 123 Efflux in Caco-2 Monolayers in the Presence of TPGS 2000

The Inhibition Protocol was performed with TPGS in which the PEG had a molecular weight of 2000.

| TPGS | | | | | |
|---|---|---|---|---|---|
| 2000 | Pₐₚₚ, A to | Pₐₚₚ, B to | Ratio, | % | % |
| Conc., | B × 10⁻⁷ | A × 10⁻⁷ | Pₐₚₚ(B − A)/ | Increase, | Decrease, |
| μmolar | cm/s | cm/s | Pₐₚₚ(A − B) | A to B | B to A |
| None | 3.62 | 64.7 | 17.9 | NA | NA |
| (Control) | | | | | |
| 33 | 5.94 | 21.7 | 3.66 | 63.7 | 66.5 |

This example shows that TPGS 2000 effectively inhibited efflux transport of Rhodamine 123 in Caco-2 monolayers.

### Example 4

### Rhodamine 123 Efflux in Caco-2 Monolayers in the Presence of TPGS-4000

The Inhibition Protocol was performed with TPGS in which the PEG had a molecular weight of 4000.

| TPGS | | | | | |
|---|---|---|---|---|---|
| 4000 | Pₐₚₚ, A to | Pₐₚₚ, B to | Ratio, | % | % |
| Conc., | B × 10⁻⁷ | A × 10⁻⁷ | Pₐₚₚ(B − A)/ | Increase, | Decrease, |
| μmolar | cm/s | cm/s | Pₐₚₚ(A − B) | A to B | B to A |
| None | 3.62 | 64.7 | 17.9 | NA | NA |
| (Control) | | | | | |
| 33 | 4.39 | 56.7 | 10.9 | 21.1 | 28.3 |

This example shows that TPGS 4000 does not effectively inhibit efflux transport of Rhodamine 123 in Caco-2 monolayers.

### Example 5

### Rhodamine 123 Efflux in Caco-2 Monolayers in the Presence of TPGS-200

The Inhibition Protocol was performed with TPGS in which the PEG had a molecular weight of 200.

| TPGS | | | | | |
|---|---|---|---|---|---|
| 200 | Pₐₚₚ, A to | Pₐₚₚ, B to | Ratio, | % | % |
| Conc., | B × 10⁻⁷ | A × 10⁻⁷ | Pₐₚₚ(B − A)/ | Increase, | Decrease, |
| μmolar | cm/s | cm/s | Pₐₚₚ(A − B) | A to B | B to A |
| None | 3.62 | 64.7 | 17.9 | NA | NA |
| (Control) | | | | | |
| 33 | 4.27 | 46.5 | 10.9 | 17.7 | 28.1 |

This example shows that TPGS 200 does not effectively inhibit efflux transport of Rhodamine 123 in Caco-2 monolayers.

### Example 6

### Rhodamine 123 Efflux in Caco-2 Monolayers in the Presence of TPGS-600

The Inhibition Protocol was performed with TPGS in which the PEG had a molecular weight of 600.

| TPGS | | | | | |
|---|---|---|---|---|---|
| 600 | Pₐₚₚ, A to | Pₐₚₚ, B to | Ratio, | % | % |
| Conc., | B × 10⁻⁷ | A × 10⁻⁷ | Pₐₚₚ(B − A)/ | Increase, | Decrease, |
| μmolar | cm/s | cm/s | Pₐₚₚ(A − B) | A to B | B to A |
| None | 3.62 | 64.7 | 17.9 | NA | NA |
| (Control) | | | | | |
| 33 | 4.76 | 23.1 | 4.85 | 31.3 | 64.3 |

This example shows that TPGS 600 does not effectively inhibit efflux transport of Rhodamine 123 in Caco-2 monolayers.

### Example 7

### Rhodamine 123 Efflux in Caco-2 Monolayers in the Presence of TPGS-3350

The Inhibition Protocol was performed with TPGS in which the PEG had a molecular weight of 3350.

| TPGS | | | | | |
|---|---|---|---|---|---|
| 3350 | Pₐₚₚ, A to | Pₐₚₚ, B to | Ratio, | % | % |
| Conc., | B × 10⁻⁷ | A × 10⁻⁷ | Pₐₚₚ(B − A)/ | Increase, | Decrease, |
| μmolar | cm/s | cm/s | Pₐₚₚ(A − B) | A to B | B to A |
| None | 3.62 | 64.7 | 17.9 | NA | NA |
| (Control) | | | | | |
| 33 | 5.26 | 37.0 | 7.04 | 45.0 | 42.8 |

This example shows that TPGS 3350 does not effectively inhibit efflux transport of Rhodamine 123 in Caco-2 monolayers.

### Example 8

### Rhodamine 123 Efflux in Caco-2 Monolayers in the Presence of TPGS-6000

The Inhibition Protocol was performed with TPGS in which the PEG had a molecular weight of 6000.

| TPGS | | | | | |
|---|---|---|---|---|---|
| 6000 | Pₐₚₚ, A to | Pₐₚₚ, B to | Ratio, | % | % |
| Conc., | B × 10⁻⁷ | A × 10⁻⁷ | Pₐₚₚ(B − A)/ | Increase, | Decrease, |
| μmolar | cm/s | cm/s | Pₐₚₚ(A − B) | A to B | B to A |
| None | 3.62 | 64.7 | 17.9 | NA | NA |
| (Control) | | | | | |
| 33 | 4.13 | 54.2 | 13.1 | 13.8 | 16.2 |

This example shows that TPGS 6000 does not effectively inhibit efflux transport of Rhodamine 123 in Caco-2 monolayers.

### Example 9

### Rhodamine 123 Efflux in Caco-2 Monolayers in the Presence of TPGS400

The Inhibition Protocol was performed with TPGS in which the PEG had a molecular weight of 400.

| TPGS | | | | | |
|---|---|---|---|---|---|
| 400 | Pₐₚₚ, A to | Pₐₚₚ, B to | Ratio, | % | % |
| Conc., | B × 10⁻⁷ | A × 10⁻⁷ | Pₐₚₚ(B − A)/ | Increase, | Decrease, |
| μmolar | cm/s | cm/s | Pₐₚₚ(A − B) | A to B | B to A |
| None | 3.62 | 64.7 | 17.9 | NA | NA |
| (Control) | | | | | |
| 33 | 4.44 | 34.7 | 7.81 | 22.4 | 46.4 |

This example shows that TPGS 400 does not effectively inhibit efflux transport of Rhodamine 123 in Caco-2 monolayers.

### Example 10

### Lack of Impact of Molecular Weight of PEG Chain on Critical Micellar Concentration of TPGS

A series of TPGS analogs were synthesized by methods similar to those used to synthesize TPGS 1000, differing only in the molecular weight of the polyethylene glycol (PEG) chain. Solutions were prepared of various known concentrations of these derivatives in water. Surface tension of these solutions were measured, and plotted against the concentration of the TPGS. These plots all showed a linear decline of surface tension with TPGS concentration, until an inflection point above which the surface tension held steady with increasing concentration of the TPGS. The concentration at this inflection point is defined as the critical micellar concentration (CMC) of the particular TPGS in water. The following is a table of the measured CMC's versus the molecular weight of the PEG chain.

| Sample | PEG MW | CMC (Wt. %) |
|---|---|---|
| TPGS 238 | 238 | 0.02 ± 0.02 |
| TPGS 600 | 600 | 0.02 ± 0.02 |
| TPGS 1000 | 1000 | 0.02 ± 0.02 |
| TPGS 2000 | 2000 | 0.02 ± 0.02 |
| TPGS 3500 | 3500 | 0.02 ± 0.02 |
| TPGS 4000 | 4000 | 0.02 ± 0.02 |
| TPGS 6000 | 6000 | 0.02 ± 0.02 |

These examples illustrate that the CMC, which is critical to the solvating power of TPGS analogs, appears comparatively independent of PEG molecular weight in the range of 200-6000.

The invention has been described in detail with particular reference to preferred embodiments thereof, but it will be understood that variations and modifications can be effected within the spirit and scope of the invention. In the drawings and specification, there have been disclosed typical preferred embodiments of the invention. Although specific terms are employed, they are used in a generic and descriptive sense only and not for purposes of limitation, the scope of the invention being set forth in the following claims.

We claim:

1. A composition comprising:
one or more lipophilic compounds,
one or more TPGS analogs that are compounds not for use as an efflux inhibitor.

2. The composition of claim 1, wherein the one or more TPGS analogs have a critical micellar concentration (CMC) that is equal to or lower than the CMC of TPGS 1000, but not more than ten times the CMC of TPGS 1000.

3. The composition of claim 1, wherein the one or more lipophilic compounds is one or more compounds for pharmaceutical use.

4. The composition of claim 1, wherein the one or more TPGS analogs have a molecular weight of at least about 10,000.

5. The composition of claim 1, wherein the one or more TPGS analogs have a molecular weight of about 400.

6. The composition of claim 1, wherein the one or more TPGS analogs have a molecular weight of less than about 550.

7. The composition of claim 1, wherein the one or more TPGS analogs have a molecular weight of at least about 2,750.

8. A method comprising coadministration to an organism of one or more lipophilic compounds and/or one or more TPGS analogs not for use as an efflux inhibitor.

9. The method of claim 8, wherein the one or more TPGS analogs have a critical micellar concentration (CMC) that is equal to or lower than the CMC of TPGS 1000, but not more than ten times the CMC of TPGS 1000.

10. The method of claim 8, wherein the one or more lipophilic compounds are for pharmaceutical use.

11. A method comprising combining one or more lipophilic compounds with one or more TPGS analogs that is a compound not for use as an efflux inhibitor.

12. The method of claim 11, wherein the one or more TPGS analogs have a critical micellar concentration (CMC) that is equal to or lower than the CMC of TPGS 1000, but not more than ten times the CMC of TPGS 1000.

13. The method of claim 11, wherein the one or more lipophilic compounds are for pharmaceutical use.

14. A method for making a composition, comprising:
providing one or more lipophilic compounds,
selecting a degree of efflux inhibition to result from the composition,
identifying one or more TPGS analogs that will cause the selected degree of efflux inhibition to result from the composition, and
combining the one or more TPGS analogs with the one or more lipophilic compounds.

15. The method of claim 14, wherein the one or more lipophilic compounds are for pharmaceutical use.

16. A composition made by the method of claim 14.

17. A method for administration to an organism, comprising:
providing one or more lipophilic compounds, selecting a degree of efflux inhibition to result from the composition,
identifying one or more TPGS analogs that will cause the selected degree of efflux inhibition to result from the composition, and
combining the one or more TPGS analogs with the one or more lipophilic compounds.

18. A container, wherein:
the contents of the container comprise one or more TPGS analogs, and the container contains, is labeled, or is otherwise accompanied by instructions to use the one or more TPGS analogs to enhance or increase solubility of a lipophilic compound or compounds in water and indicates in any manner that the TPGS or TPGS composition does not inhibit effect on efflux or has a diminished, limited, or insignificant inhibitory effect on efflux.

19. A method comprising:
providing one or more lipophilic compounds,
selecting a degree of efflux inhibition to result from the composition,
identifying one or more TPGS compounds that will cause the selected degree of efflux inhibition to result from the composition, and
coadministering the one or more TPGS compounds and the one or more lipophilic compounds to a human or animal.
